(19) United States
(12) Patent Application Publication
(10) Pub. No.: US 2011/0257649 A1
(43) Pub. Date: Oct. 20, 2011

(54) Electrode For An Electrophysiological Ablation Catheter

(75) Inventors: Wolfgang Geistert, Rheinfelden (DE); Martin Erben, Berlin (DE); Andreas Kiefer, Loerrach (DE); Ralf Kaufmann, Loerrach (DE)

(73) Assignee: VASCOMED GMBH, Binzen (DE)

(21) Appl. No.: 13/080,078

(22) Filed: Apr. 5, 2011

Related U.S. Application Data
(60) Provisional application No. 61/325,825, filed on Apr. 20, 2010.

(51) Int. Cl. A61B 18/18; A61B 5/04

(52) U.S. Cl. 606/41; 600/374

(57) ABSTRACT

An electrode for an electrophysiological ablation catheter including an electrode body extending along a longitudinal axis, the electrode body including an electrode outer surface for emitting high-frequency signals and/or for measuring physiological signals, a first attachment point on a first end, at which the electrode is attached to a first catheter shaft, an irrigation lumen extending parallel to the longitudinal axis and through which cooling agent may be directed out of the first catheter shaft and into the electrode, and which forms an opening at the first end of the electrode body, the opening connected to a lumen of the first catheter shaft, and at least one cooling-agent passage connected to the irrigation lumen, the cooling-agent passage situated at an angle to the longitudinal axis and forming first and second openings in the electrode outer surface, through which the cooling agent may be released into the surroundings, as cooling-agent flow.

### CROSS-REFERENCE TO RELATED APPLICATIONS

This patent application claims the benefit of co-pending U.S. Provisional Patent Application No. 61/325,825, filed on Apr. 20, 2010, which is hereby incorporated by reference in its entirety.

### TECHNICAL FIELD

The invention relates to electrodes and, more particularly, to an electrode for an electrophysiological ablation catheter.

### BACKGROUND

Atrial fibrillation is the most common form of cardiac arrhythmia, affecting approximately one million individuals, mostly elderly, in Germany alone. Experts estimate that the number of individuals who are affected will increase to 2.5 million by the year 2050. Abnormal cardiac rhythm may be caused by general health problems or heart disease, but also by stress, alcohol, caffeine, serious infections, or medication.

Atrial fibrillation means that the atria of the heart functions irregularly and at a frequency of more than 300 beats per minute. It occurs when the electrical signals are not emitted not only from the sinoatrial node, the heart's natural pacemaker, but rather also from other sites of origin (foci) which are usually located in the pulmonary veins. As a result, circulating electrical stimulations are triggered in the atria. The atrioventricular node (AV node), which transmits the signals that originate in the sinoatrial node and travel through the atria to the chambers of the heart (ventricles), usually limits the number of impulses for the most part and, therefore, the entire heart normally does not beat at this rapid frequency. However, due to the aberrant stimulus, the cardiac muscle does not have enough time to adequately contract in order to initiate the next pumping action. As a consequence, less blood and, therefore, oxygen from the atria, reaches the ventricles and, from here, the systemic circulation. In approximately 80 percent of patients, the reduced cardiac pumping capacity results in a restriction of physical capability due to, for example, palpitations, shortness of breath, dizziness or fear, and results in diminished quality of life. If atrial fibrillation persists, the patient is at higher risk of stroke since, due to the diminished cardiac pumping capacity, blood clots may form in the left atrium and reach the brain.

Ablation is a therapy regimen that can permanently cure atrial fibrillation. In this regimen, the region of the heart that causes or promotes the arrhythmia is thermally destroyed (ablated) via energy output. Ablation destroys the focus or foci and isolates the conducting cardiac tissue via barriers composed of scar tissue, which is not electrically conductive. Since the abnormal electrical signals are now no longer able to reach the atrium, it is only the sinoatrial node that determines the beat, as nature intended, and natural cardiac rhythm is restored. Many episodes of atrial fibrillation are not triggered by individual points but, rather, by several sites of origin. Physicians typically isolate these sites of origin using ablation lines that subdivide the atria into interconnected corridors and dead ends and, therefore, the electrical impulses now only follow the specified paths of conduction. Various methods that use different forms of energy are available for ablation. One of the most frequently used forms of energy is high-frequency current using minimally invasive catheter ablation carried out using an electrophysiological catheter.

When minimally invasive catheter ablation is performed by a physician, in particular an electrophysiologist, an electrophysiological ablation catheter is introduced into a vein—usually in the inguinal region—and advanced to the heart. Next, the catheter tip is brought into direct contact with the cardiac tissue and emits high-frequency energy in order to destroy the cardiac tissue. “Mapping” is used to substantially simplify the planning and implementation of catheter ablation. In this electrophysiological examination, a three-dimensional image of the conduction of stimulus in the atrium is created, thereby making it possible to navigate the electrophysiological ablation catheter to the exact point. It also reduces radiation exposure caused by conventional fluoroscopy. Ablation can completely eliminate atrial fibrillation by a high percentage, and so patients are largely relieved of symptoms after a certain period of time. It is advantageous that, even though ablation may occasionally take longer when carried out using an electrophysiological ablation catheter, the patient does not have to undergo a stressful surgical operation.

An electrophysiological ablation catheter of this type is typically composed of an elongated catheter shaft that includes a plurality of lumina, usually including a lumen for control means such as, for example, puller wires, and a lumen in which signal lines are guided to electrodes at the distal end. The signal lines are guided in an insulated manner, and are used to measure body signals and/or to transmit high-frequency signals in order to generate ablation energy. The electrodes may be one or more ring electrodes on the catheter shaft, or a top electrode that is located on the distal end of the catheter shaft. The proximal end, which is opposite the distal end, is not introduced into the body, and also usually includes control means for use by the electrophysiologist to actively control the distal catheter shaft, and includes connections for reversible connection to measurement devices, high-frequency (HF) generators and/or cooling fluid pumps, or combinations thereof. The catheter shaft may include various sections made of different materials and/or having different hardnesses that are advantageous in terms of controllability. A catheter of this type is presented in U.S. Pat. No. Re. 34,502, as an example.

In one embodiment, a catheter of this type may also be used for cooling. In one variant which is designed as closed cooling, the top electrode includes an internal chamber, into which a cooling agent from the proximal end is directed into the catheter shaft, via an additional lumen. This medium is used to dissipate heat via a further lumen in the catheter shaft, direct it back to the proximal end, and release it.

Another variant is designed as “open cooling”, and is also referred to as irrigation catheters. In this variant, the top electrode includes openings in the distal end of the catheter shaft, through which the cooling agent, which has passed through the irrigation lumen, may emerge.

FIGS. 1A and 1B show a first embodiment of a known top electrode of this type that includes openings (“irrigation openings”) for an electrophysiological ablation catheter with open cooling. Top electrode 1 is formed by a metal sleeve 2, in which openings 3 are provided. The metal sleeve encloses an internal chamber 9. Openings 3 may be created, e.g., using metal-removing methods or other methods, such as lasers. Top electrode 1 is fastened to the distal end of an elongated catheter shaft 4 that is suited for introduction into a corporeal lumen. Catheter shaft 4 includes a plurality of lumina, such as a lumen for control wires, or a lumen 7 for signal lines, which are guided in an insulated manner, for the measurement of body signals and/or for the transmission of high-frequency signals in order to generate ablation energy at the top electrode 1. Furthermore, catheter shaft 4 includes an irrigation lumen 5, through which a cooling agent, such as, for example, normal saline solution, is conveyed to top electrode 1. The cooling agent is continually supplied at a pressure and in a quantity such that it fills the internal chamber 9 of the top electrode 1 and is dispensed through openings 3, out of internal chamber 9, and into the surroundings, in order to ensure cooling, e.g., of cardiac tissue. To ensure proper cooling agent pressure and quantity, the electrophysiological catheter is connected, e.g., to a cooling fluid pump (not shown) at its proximal end that faces away from the top electrode 1. A pump of this type, and a generator for generating the high-frequency energy are described, e.g., in U.S. Publication No. 2009/0187186, the entire scope and contents of which are incorporated into this patent application by reference in its entirety. When the cooling agent emerges from openings 3, it forms a cooling flow 6 that is oriented substantially orthogonally to the outer top electrode 1 surface that encloses the opening 3. An optional temperature sensor, which may be guided in lumen 7 or in a further lumen, is not shown. Given that it is filled approximately completely with cooling agent, this top-electrode configuration creates good cooling properties in the electrode itself. The cooling effect on the surrounding tissue is inadequate, however, since the cooling flows, which emerge orthogonally to the top electrode 1 surface, are incapable of adequately cooling all outer surfaces of the top electrode 1. This applies, in particular, for the transition regions between the catheter shaft 4 and the top electrode 1. Due to the edge effect, current density is high in these transition regions when HF ablation energy is output, and a greater amount of heat is therefore generated. As a result, the risk of unwanted clot formation is particularly high in these transition regions.

FIGS. 2A and 2B show a further top electrode, which is known from the prior art, for an electrophysiological catheter that has the same shaft design as that described above. However, the top electrode 1 is formed by a solid metal element 10, in which passages 11 with openings 12 are provided, through which the cooling agent may emerge into the catheter surroundings. The passages, preferably six in all, intersect irrigation lumen 5 orthogonally at its end. The cooling agent is continually supplied at a pressure and in a quantity such that it emerges from the openings 12 of passages 11 under a certain pressure, thereby creating a cooling flow 6 and ensuring cooling, e.g., of cardiac tissue. In this known embodiment as well, cooling flow 6 is oriented substantially orthogonally to the outer top surface electrode 1 surface that encloses the openings 12. This embodiment likewise has the problem that, due to the orthogonality of the cooling flow relative to the outer top electrode 1 surface, the cooling effect on the surrounding tissue is inadequate. This applies in particular for the boundary regions located between the catheter shaft 4 and the top electrode 1.

The disclosed electrode is directed at overcoming one or more of the above-identified problems.

### SUMMARY

The problem addressed by the present invention is thus that of designing the cooling of the electrodes, in particular, the top electrode of an electrophysiological ablation catheter, to be more effective, and of preventing coagulations of the corporeal medium surrounding the electrodes.

This problem is solved by an electrode and by an electrophysiological ablation catheter according to the claims.

The present invention is based on the finding that the irrigation solutions known from the prior art, which have angles of approximately 90-degrees relative to the surface of the electrode, are inadequate in terms of ensuring complete and effective coverage of the outer electrode surface with a cooling agent and, therefore, of ensuring effective cooling during the output of the high-frequency ablation signal. In particular, it has been shown that the solutions made available in the prior art are incapable of covering the transition between the catheter shaft material and the electrode material in a manner such that cooling of explicitly this catheter section is ensured and the repeated occurrence of coagulations is prevented.

The present invention therefore relates to an electrode for an electrophysiological ablation catheter comprising an electrode body that extends along a longitudinal axis, in which the electrode body includes an electrode outer surface for emitting high-frequency signals and/or for measuring physiological signals, a first attachment point on a first end, at which the electrode is attached to a first catheter shaft, an irrigation lumen that extends parallel to the longitudinal axis and through which a cooling agent may be directed out of the first catheter shaft and into the electrode, and which forms an opening at the first end of the electrode body, the opening being connected to a lumen of the first catheter shaft, and at least one cooling-agent passage that is connected to the irrigation lumen, the cooling-agent passage being situated at an angle to the longitudinal axis and forming a first opening and a second opening in the electrode outer surface, through which the cooling agent may be released into the surroundings, as cooling-agent flow.

The present invention is characterized, in particular, by the fact that the degree measure of the angle relative to the longitudinal axis is such that the cooling-agent passage includes an extension component parallel to the longitudinal axis of the electrode and, therefore, the cooling-agent flow emerging from the at least one opening includes an extension component along the electrode outer surface and spreads out in a manner such that the electrode and its immediate vicinity are cooled. This means that, when the cooling agent emerges from the openings of the cooling-agent passages, the cooling-agent flow maintains a direction along an extension component in the direction of the longitudinal axis that extends beyond the boundary of the electrode, that is, in the direction toward the first catheter shaft and away from the electrode, in the direction of the tissue to be ablated, and primarily past the attachment point on the first end of the electrode body to the first catheter shaft, where coagulations are likely to occur. Particularly good coverage with the cooling agent therefore takes place.

In a special embodiment, the cooling-agent passage extends completely through the electrode and, therefore, the cooling-agent passage includes a second opening in the electrode outer surface that is diametrically opposite the first opening.

To supply the cooling-agent passages, the irrigation lumen forms an opening in the first end of the electrode body, which is connected to a lumen of the first catheter shaft. The electrode outer surface may include, in the region of the first and/or second openings, funnel-shaped indentations or a radially circumferential groove, to ensure that the cooling medium spreads along the attachment point toward the first catheter shaft.

The at least one cooling-agent passage passes diagonally through the rotationally-symmetrical electrode body in a manner such that the irrigation lumen and the at least one cooling-agent passage are connected. In this sense, “diagonally” means that the at least one cooling-agent passage may intersect the cylindrical plan of the electrode body—which is circular as viewed from the first end—as a secant. In this case, the irrigation lumen is located parallel to the longitudinal axis of the electrode body. However, if the irrigation lumen is located on the longitudinal axis, the cooling-agent passages form a diameter which is also covered by the term “diagonal” or “diagonal”, thereby creating a connection to the irrigation lumen. Notwithstanding this, the at least one cooling-agent passage includes an extension component in the direction of the longitudinal axis, that is, “diagonal” only refers to a plan, and preferably a rotationally symmetrical, circular plan.

The direction of the extension in the longitudinal axis is defined in that the at least one cooling-agent passage includes a section that has an extension component parallel to the longitudinal axis, in the direction of the first end of the electrode body and, therefore, the first opening is formed in the electrode outer surface in the vicinity of, or at, the first attachment point, and so the cooling-agent flow is diverted in the direction of the first attachment point, thereby cooling the electrode and the first catheter shaft. The angle between the stated section of the cooling-agent passage and the longitudinal axis is between 1 and 80 degrees, and preferably between 30 and 60 degrees. As a result, the cooling flow that emerges from the openings in the electrode outer surface maintains the decisive direction, which points in the direction of the catheter shaft, and may therefore prevent an impermissible warming of the boundary region and coagulation of bodily fluids. It is therefore ensured that the cooling medium does not emerge in the orthogonal direction relative to the longitudinal axis of the electrode body.

The connection between the irrigation lumen and the at least one cooling-agent passage is preferably located on a plane between the first end and a second end, which faces away from the first end, and preferably located half-way between the first end and the second end.

According to a variant of the present invention, the electrode is designed as a ring electrode. This means that this electrode is not a terminal electrode, and it is possible for further electrodes to be located on a first catheter shaft, connected in front of or behind this ring electrode. According to this variant, the electrode body includes a second attachment point for a second catheter shaft on a second end. In a preferred embodiment—if a plurality of cooled ring electrodes is present—the irrigation lumen forms an opening in the second end of the electrode body that is connected to a lumen of the second catheter shaft. A further electrode, according to the present invention, may be attached to this second shaft, on the opposite end and, therefore, the second catheter shaft performs the same function, relative to this further electrode, as the first catheter shaft relative to the initially mentioned electrode. Advantageously, a plurality of electrodes of this type may perform various tasks and improve the success of measurement and therapy.

In the case of such a variant of the present invention, the cooling-agent passage is advantageously designed such that the second opening of the cooling-agent passage is diametrically opposite the first opening in the electrode outer surface, in the vicinity of or at the second attachment point. The second attachment point is therefore likewise subjected to optimal cooling.

In all embodiments, the cooling-agent passage need not extend along an axis but, rather, may instead be “bent”. For example, the connection between the irrigation lumen and a first section of the cooling-agent passage may absolutely be approximately orthogonal to the longitudinal axis, or it may form a shallow angle (for example, 45° to 80°) with the longitudinal axis. Connected thereto, the cooling-agent passage extends in the direction of the electrode outer surface at an acute angle of approximately 1° to 45°, in order to perform the desired functionality of cooling the attachment point between the first end of the electrode and the catheter shaft.

According to a further variant of the present invention, the electrode may be designed as a top electrode. In this case, the electrode forms the outermost end of a catheter, which may be introduced into a body. This electrode is characterized by the fact that the electrode includes a second end that faces away from the first end of the electrode body, the second end preferably having an atraumatic shape, and, particularly preferably, a hemispherical, trapezoidal, or rounded shape, and forms an electrode outer surface as the electrode top surface. This simplifies the atraumatic introduction of the catheter into the corporeal lumen, and may also prevent the accidental perforation of the body tissue to be treated, such as, for example, the endocardium.

In this variant of the electrode, the second openings of the cooling-agent passages are formed in the electrode top surface. As a result, the “tip” of the catheter, which is formed by the spherical and, therefore, atraumatic end of the electrode, and which is typically essential to the punctiform destruction of body tissue, is likewise cooled in a manner such that the tissue is not damaged in a manner that is dangerous to health, and such that coagulations do not occur.

The present invention relates, in a further aspect, to an electrophysiological ablation catheter that includes an elongated first catheter shaft that has a proximal end and a distal end, an electrode, which was described above and is attached to the distal end of the catheter shaft, at least one lumen that is located in the elongated catheter shaft and extends from the proximal end to the distal end of the catheter shaft, at least one of which is connected at its distal end to the irrigation lumen of the stated electrode according to the present invention, and is connected at its proximal end to a connection for supplying the cooling agent, and at least one electrical signal line for the transmission of high-frequency signals and/or for the measurement of physiological signals at the above-described electrodes. This signal line likewise extends from the proximal end of the catheter, where a connection is located for forwarding the measurement signals or for supplying high-frequency signals. The line may extend in one of the stated lumina, e.g., to ensure cooling of the signal line, e.g., under the influence of electromagnetic radiation. A solution of this type is shown in U.S. Pat. No. 7,507,237, the entire scope of which is incorporated in this application. As an alternative, the signal line may be embedded in the shaft material.

Optionally, although not mandatory for every embodiment, the electrophysiological ablation catheter may include control means which are located in and at the proximal end of the catheter shaft. As mentioned above, these control means may be puller wires or puller cables which are fastened distally to the distal end of the catheter shaft or to the electrode according to the present invention, and which may be controlled proximally using a known control handle in order to attain a bending of the distal region of the electrophysiological catheter.

Furthermore, a catheter of this type may include one or more various sensors in or on the electrode, e.g., a temperature sensor for measuring temperature during treatment, or pressure sensors for measuring the contact pressure of the catheter against the tissue. As stated above, the measurement signals of these sensors may preferably be transmitted to the proximal end, or in separate measurement lines.

A catheter according to the present invention preferably includes a ring electrode and a top electrode.

Further details of the present invention, which have not been stated, are stated in the description.

Various other objects, aspects and advantages of the present invention can be obtained from a study of the specification, the drawings, and the appended claims.

### DESCRIPTION OF THE DRAWINGS

In the drawings:

FIG. 1A shows an exterior view of a first embodiment, which is known from the prior art, of a top electrode, including internal chamber and irrigation openings.

FIG. 1B shows a longitudinal section of a first embodiment, which is known from the prior art, of a top electrode, including internal chamber and irrigation openings.

FIG. 2A shows the exterior view of a further top electrode, which is known from the prior art, including passages to the irrigation openings.

FIG. 2B shows a longitudinal section of a further top electrode, which is known from the prior art, including passages to the irrigation openings.

FIG. 3A shows an exterior view of a top electrode according to the present invention.

FIG. 3B shows a longitudinal section of a top electrode according to the present invention.

FIG. 4A shows an exterior view of a top electrode according to the present invention, which has been inserted into the shaft of the first catheter.

FIG. 4B shows a longitudinal view of a top electrode according to the present invention, which has been inserted into the shaft of the first catheter.

FIG. 5A shows an exterior view of a ring electrode according to the present invention.

FIG. 5B shows a longitudinal section of a ring electrode according to the present invention.

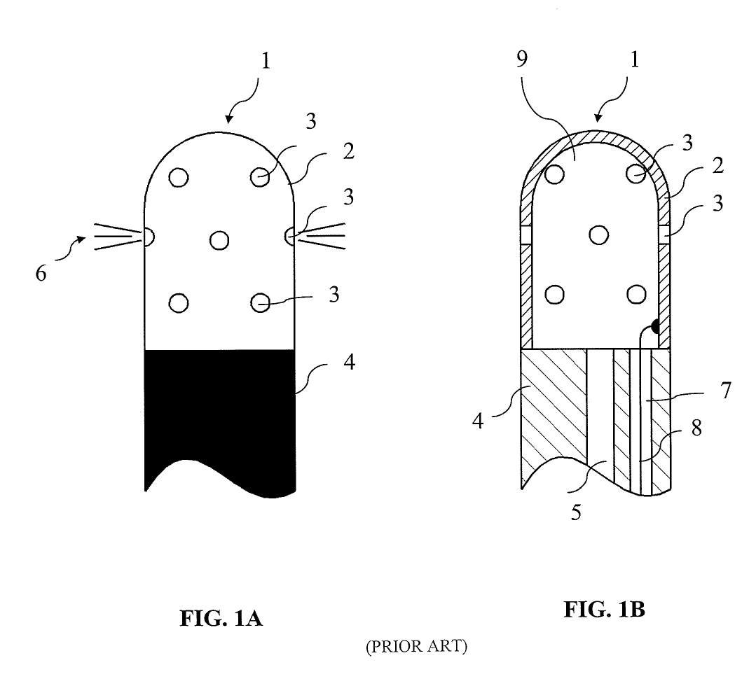

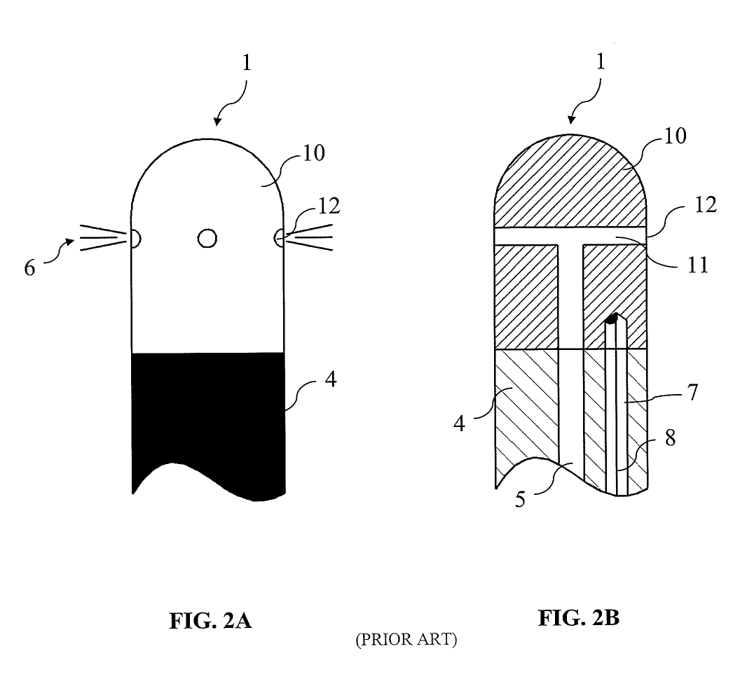

### DETAILED DESCRIPTION

The present invention is explained/described below with reference to an embodiment of a top electrode. Of course, the present invention may also relate to any electrode shape, such as, but not limited to, ring electrodes.

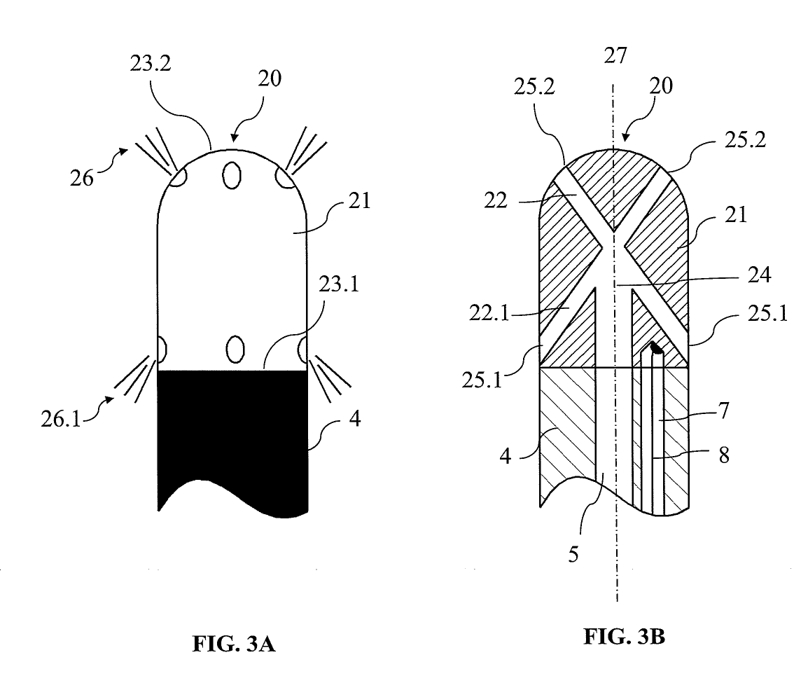

FIGS. 3A and 3B are schematic depictions of a first embodiment of the top electrode for an electrophysiological catheter that has the same shaft design as that described above. Top electrode 20 is formed by a solid electrode body 21 made of, for example, metal, in which diametral cooling-agent passages 22, 22.1 are provided. The top electrode 20 includes a first end 23.1, which is proximal in this case, at which an attachment point for fastening to catheter shaft 4 is located. The cooling agent (e.g., normal saline solution) is conducted via a lumen 5 in the catheter shaft 4 through an irrigation lumen 24 into the top electrode 20, fills passages 22, 22.1, and emerges from the openings 25. One part 26.1 of the cooling flow 26 is directed toward the proximal end of top electrode 20, i.e. toward the point of attachment to catheter shaft 4. This is realized by the fact that a section 22.1 of cooling-agent passage 22 includes an extension component parallel to the longitudinal axis 27 in the direction toward first end 23.1 of the electrode body, and therefore forms an opening 25.1 in the electrode outer surface in the vicinity of, or at, the first attachment point. In a further lumen 7, electrical supply line 8 is advanced toward top electrode 20. An optional temperature sensor, which may be guided in the same lumen or in another lumen, is not shown.

Second end 23.2, which is the distal end in this case, is generally spherical in shape, and outlet openings 25.2 in this electrode top surface cause the cooling flow to likewise contribute to cooling at this point.

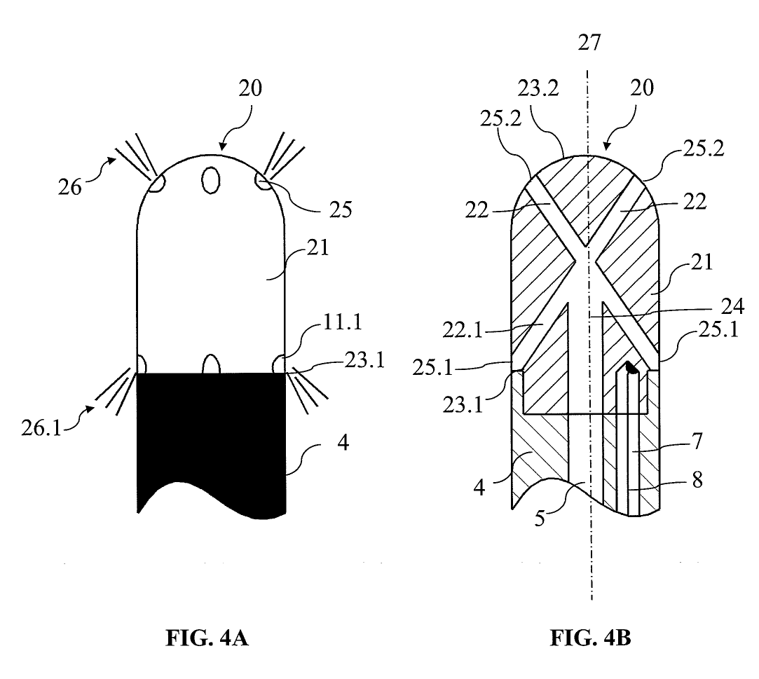

FIGS. 4A and 4B are schematic depictions of a second embodiment of the top electrode for an electrophysiological catheter that has the same shaft design as that described above. Identical or similar parts are labeled with the same reference numerals used in FIGS. 3A and 3B, and they will not be explained again here. The top electrode 20 is introduced into the distal end of catheter shaft tube 4 in a manner such that a part 25.1 of openings 25 of the cooling-agent passages 22, 22.1 is located exactly at the attachment point on first end 23.1 of the electrode body, that is, directly on the outer surface of the top electrode 20 toward the outer surface of the catheter shaft tube which has the same outer diameter as the electrode.

In an improved variant of this embodiment, part 25.1 of openings 25 may be widened at the top electrode/shaft transition, or may be connected to a groove that is circumferential at the transition, in order to optimally distribute the cooling fluid at the transition.

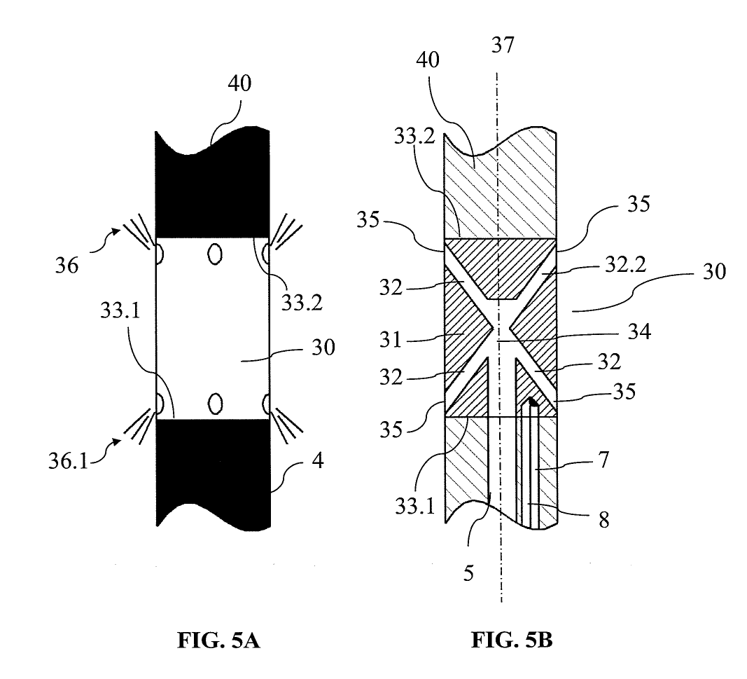

FIGS. 5A and 5B are schematic depictions of a first embodiment of a ring electrode for an electrophysiological catheter that has the same shaft design as that described above. Identical or similar parts are labeled with the same reference numerals used in FIGS. 3A, 3B, 4A, and 4B, and they will not be explained again here. For example, cooling-agent passages 32 have the same features as cooling-agent passages 22, and the openings 35 have the same properties as the openings 25 in the top electrode 20 as described in FIGS. 3A, 3B, 4A, and 4B. Likewise, lumina 5 and 7, and supply line 8 perform the same functions.

In this case, the ring electrode includes, at second end 33.2, a second point of attachment for a second catheter shaft 40. In this embodiment, openings 35, which form cooling-agent passages 32 with the electrode outer surface, all lie on the same electrode outer surface and are used to cool the attachment point at ends 33.1 and 33.2, to thereby prevent coagulations.

In an improved variant of this embodiment, the openings 35 may be widened at the first and second attachment points, or may be connected to a groove that is circumferential at the transitions, in order to optimally distribute the cooling fluid at the transitions.

According to a further embodiment, which is not specifically depicted in the drawings but will be apparent from the description herein, the second catheter shaft 40 has the same features in terms of lumina 5 and 7, and supply line 8 as in the catheter shaft 4. In fact, the supply line 8 is guided within lumen 7 through the electrode in an insulated manner, while lumen 5 continues toward second end 33.2 of the electrode body in a manner such that it forms a second opening which is connected to the lumen, which is not shown, in catheter shaft 40. This makes it possible to attach a plurality of cooled ring electrodes in series.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. An electrode for an electrophysiological ablation catheter that includes an electrode body that extends along a longitudinal axis, the electrode comprising:
an electrode outer surface for emitting high-frequency signals and/or for measuring physiological signals;
a first attachment point on a first end, at which the electrode is attached to a first catheter shaft, through which signals and cooling agent may be directed to the electrode;
an irrigation lumen that extends parallel to the longitudinal axis of the electrode body, through which the cooling agent may be directed out of the first catheter shaft and into the electrode, and which forms an opening at the first end of the electrode body, the opening being connected to a lumen of the first catheter shaft; and
at least one cooling-agent passage that is connected to the irrigation lumen and is situated at an angle to the longitudinal axis, the cooling-agent passage forming at least one opening in the electrode outer surface, through which the cooling agent may be released into the surroundings, as cooling-agent flow,
wherein the degree measure of the angle is such that the cooling-agent passage includes an extension component parallel to the longitudinal axis of the electrode body, such that the cooling-agent flow that emerges from the at least one opening includes an extension component along the electrode outer surface and spreads out in a manner such that the electrode and its immediate vicinity are cooled.

2. The electrode as recited in claim 1, wherein the at least one cooling-agent passage passes diagonally through the electrode body in a manner such that the irrigation lumen and the at least one cooling-agent passage are connected.

3. The electrode as recited in claim 1, wherein a section of the cooling-agent passage includes an extension component parallel to the longitudinal axis of the electrode body, in the direction of the first end of the electrode body, thereby forming a first opening in the electrode outer surface in the vicinity of, or at, the first attachment point, such that the cooling-agent flow is diverted in the direction toward the first attachment point, thereby cooling the electrode and the first catheter shaft.

4. The electrode as recited in claim 3, wherein the section of the cooling-agent passage that includes an extension component parallel to the longitudinal axis of the electrode body, in the direction toward the first end of the electrode body, forms an angle with the longitudinal axis of the electrode body having a degree measure between 1° and 80°.

5. The electrode as recited in claim 3, wherein the section of the cooling-agent passage that includes an extension component parallel to the longitudinal axis of the electrode body, in the direction toward the first end of the electrode body, forms an angle with the longitudinal axis of the electrode body having a degree measure between 30° and 60°.

6. The electrode as recited in claim 1, wherein the cooling-agent passage includes a second opening in the electrode outer surface that is diametrically opposite the first opening.

7. The electrode as recited in claim 1, wherein the electrode outer surface includes, in the region of the first and/or second openings, funnel-shaped indentations or a radially circumferential groove, to ensure that the cooling medium spreads along the attachment point toward the first catheter shaft.

8. The electrode as recited in claim 1, wherein the electrode body includes a second end, which faces away from the first end, and the connection between the irrigation lumen and the at least one cooling-agent passage is located on a plane between the first end and the second end.

9. The electrode as recited in claim 1, wherein the electrode body includes a second end, which faces away from the first end, and the connection between the irrigation lumen and the at least one cooling-agent passage is located on a plane between the first end and the second end half-way between the first end and the second end.

10. The electrode as recited in claim 1, wherein the electrode body includes a second attachment point on a second end for a second catheter shaft, and, on the second end of the electrode body, the irrigation lumen forms an opening that is connected to a lumen of the second catheter shaft.

11. The electrode as recited in claim 10, wherein the second opening of the cooling-agent passage is diametrically opposite the first opening in the electrode outer surface, in the vicinity of, or at, the second attachment point.

12. The electrode as recited in claim 1, wherein the electrode comprises a top electrode that includes a second end that faces away from the first end of the electrode body, the second end preferably having an atraumatic shape and forming an electrode outer surface as the electrode top surface.

13. The electrode as recited in claim 12, wherein the atraumatic shape comprises a hemispherical, trapezoidal, or rounded shape.

14. The electrode as recited in claim 12, wherein the second opening of the cooling-agent passage is formed in the electrode top surface.

15. An electrophysiological ablation catheter comprising:
an elongated first catheter shaft that includes a proximal end and a distal end;
an electrode, as recited in claim 1, that is attached to the distal end of the elongated first catheter shaft;
at least one lumen that is located in the elongated first catheter shaft and extends from the proximal end to the distal end, at least one of the lumina being connected, at its distal end, to the irrigation lumen of the electrode as recited in claim 1, and, at its proximal end, to a connection for supplying the cooling agent; and
at least one electrical signal line for the transmission of high-frequency signals and/or for the measurement of physiological signals at the electrode as recited in claim 1, which extends from the proximal end of the elongated first catheter shaft to the electrode.

16. The electrophysiological ablation catheter as recited in claim 15, wherein the catheter includes at least one ring electrode.

17. The electrophysiological ablation catheter as recited in claim 16, wherein the electrode body includes a second attachment point on a second end for a second catheter shaft, and, on the second end of the electrode body, the irrigation lumen forms an opening that is connected to a lumen of the second catheter shaft.

18. The electrophysiological ablation catheter as recited in claim 15, wherein the catheter includes at least one top electrode

19. The electrophysiological ablation catheter as recited in claim 18, wherein the electrode comprises a top electrode that includes a second end that faces away from the first end of the electrode body, the second end preferably having an atraumatic shape and forming an electrode outer surface as the electrode top surface.

20. The electrode as recited in claim 19, wherein the atraumatic shape comprises a hemispherical, trapezoidal, or rounded shape.
